(19) United States
(12) Patent Application Publication
(10) Pub. No.: US 2015/0175553 A1
(43) Pub. Date: Jun. 25, 2015

(54) PRODUCTION OF 18F-LABELLED COMPOUNDS COMPRISING HYDROLYTIC DEPROTECTION STEP AND SOLID PHASE EXTRACTION

(71) Applicant: GE HEALTHCARE LIMITED, BUCKINGHAMSHIRE (GB)

(72) Inventors: Ludovic Wouters, Battice (BE); Steve Lignon, Loncin (BE)

(73) Assignee: GE HEALTHCARE LIMITED, BUCKINGHAMSHIRE (GB)

(21) Appl. No.: 14/356,766

(22) PCT Filed: Nov. 29, 2012
(86) PCT No.: PCT/EP2012/073926

§ 371 Date: May 7, 2014

Related U.S. Application Data
(60) Provisional application No. 61/564,880, filed on Nov. 30, 2011.

(30) Foreign Application Priority Data
Nov. 30, 2011 (GB) 1120586.1

(51) Int. Cl. C07D 233/91; B01J 19/00; A61K 51/04

(52) U.S. Cl. CPC C07D 233/91; A61K 51/0453; B01J 19/004; C07B 2200/05

(57) ABSTRACT

The present invention provides a simplified method for the preparation of ¹⁸F-labelled compounds that is particularly suitable for automation. The method of the invention is specifically applicable where the ¹⁸F-labelled compound is prepared from a labelling precursor that comprises protecting groups and wherein the synthetic route to the final compound includes removal of these protecting groups by acid or alkaline hydrolysis. Also provided by the present invention is a cassette useful for carrying out the method of the invention in an automated manner.

### TECHNICAL FIELD OF THE INVENTION

The present invention relates to a method for the synthesis of ¹⁸F-labelled compounds and in particular ¹⁸F-labelled compounds that are useful as positron emission tomography (PET) tracers.

### DESCRIPTION OF RELATED ART

The radioisotopes suitable for detection in positron emission tomography (PET) have notably short half-lives. Carbon-11 (¹¹C) has a half-life of about 20 minutes, nitrogen-13 (¹³N) has a half-life of about 10 minutes, oxygen-15 (¹⁵O) has a half-life of about 2 minutes and fluorine-18 (¹⁸F) has a half-life of about 110 minutes. Synthetic methods for the production of compounds labelled with these radionuclides need to be as quick and as high yielding as possible. This is particularly important in the case of compounds destined to be used for in vivo imaging, commonly known as PET tracers. Furthermore, the step of adding the radioisotope to the compound should be as late as possible in the synthesis, and any steps taken following the addition of radioisotope for the work up and purification of the radioisotope-labelled compounds should be completed with as little time and effort as possible.

PET tracers, and [¹⁸F]-radiotracers in particular, are now often conveniently prepared by means of an automated radiosynthesis apparatus, e.g. Tracerlab™ and Fastlab™ from GE Healthcare Ltd. A disposable cassette in which the radiochemistry is performed is fitted to the apparatus. The cassette normally includes fluid pathways, a reaction vessel, and ports for receiving reagent vials as well as any solid phase extraction (SPE) cartridges used in post-radiosynthetic clean up steps. A well-developed automatic synthesis method provides advantages of speed, convenience and a generally reliable routine supply of the PET tracer. Furthermore and importantly, radiation burden to the operator is reduced to a minimum.

The synthesis of a number of ¹⁸F-labelled PET tracers comprises ¹⁸F labelling of a protected precursor compound, with subsequent removal of the protecting groups by acidic or alkaline hydrolysis. Examples of such ¹⁸F-labelled PET tracers include ¹⁸F-fluorodeoxyglucose (¹⁸F-FDG), 6-[¹⁸F]-L-fluorodopa (¹⁸F-FDOPA), ¹⁸F-fluorothymidine (¹⁸F-FLT), 1-H-1-(3-[¹⁸F]fluoro-2-hydroxypropyl)-2-nitroimidazole (¹⁸F-FMISO), ¹⁸F-1-(5-fluoro-5-deoxy-α-arabinofuanosyl)-2-mitroimidazole (¹⁸F-FAZA), 16-α-[¹⁸F]-fluoroestradiol (¹⁸F-FES), and 6-[¹⁸F]-fluorometarminol (¹⁸F-FMR) (see for example chapters 6 and 9 of “Handbook of Radiopharmaceuticals” 2003; Wiley: by Welch and Redvanly, and chapter 8 of “Basics of PET Imaging, 2^{nd} Edition” 2010; Springer: by Saha).

Taking [¹⁸F]FMISO as an example, Oh et al (2005 Nuc Med Biol; 32: 899-905) describes an automated method for its synthesis. On a TracerLab Mx [¹⁸F]FDG synthesis module (GE Healthcare) and using modified disposable [¹⁸F]FDG cassettes, a solution of the precursor compound 1-(2′-nitro-1′-imidazolyl)-2-O-tetrahydrofuranyl-3-O-toluenesulfonylpropanediol in acetonitrile (MeCN) was reacted with [¹⁸F]fluoride (¹⁸F−) at 95-120° C. for 300-600 seconds and at 75° C. for 280 seconds, then hydrolysed at 105° C. for 300 seconds with 1N HCl following solvent removal, and neutralised using NaOH. The neutralised [¹⁸F]FMISO crude solution was purified using high-performance liquid chromatography (HPLC) to result in [¹⁸F]FMISO having decay-corrected end of synthesis (EOS) radiochemical yields of 58.5±3.5%. The reported synthesis time was 60.0±5.2 minutes.

Frank et al (2009 Appl Radiat Isotop; 67(6): 1068-1070) report the synthesis of [¹⁸F]FMISO using an automated synthesiser. The precursor compound 1-(2′-nitro-1′imidazolyl)-2-O-tetrahydropyranyl-3-O-toluenesulfonylpropanediol (NITTP) was labelled with ¹⁸F⁻ in acetonitrile at 120° C. for 10 minutes, deprotected with 1N HCl at 105° C. for 5 minutes and neutralised with 1N NaOH. The neutralised crude product reaction mixture was purified using HPLC. The decay-corrected yields were reported to be 20-30%.

The above-described automated methods for the production of [¹⁸F]FMISO both use purification by HPLC. It is preferred that a purification method taking up less time and space is used, such as solid-phase extraction (SPE). Chang et at (2007 App Rad Isotop; 65: 682-686) describe an automated method for the synthesis of [¹⁸F]FMISO using a Scanditronix Anatech RB III robotic system. The precursor compound (2′-nitro-1′-imidazolyl)-2-O-acetyl-3-O-tosylpropanol in acetonitrile was labelled with ¹⁸F⁻ at 95° C. for 10 minutes, hydrolysed using 1N HCl at 90° C. for 10 minutes following solvent removal and neutralised with a solution of NaOH. The neutralised crude reaction product was purified by first passing through a C18 Sep-Pak cartridge and then a neutral alumina Sep-Pak cartridge. The uncorrected EOS radiochemical yields reported were 30±5%, and the synthesis time was 65 minutes. Radiochemical yield was reduced and no apparent advantage in synthesis time was provided by this method as compared with the earlier method including HPLC purification disclosed by Oh et al (referenced above).

There is therefore scope for the provision of an automated method for the production of [¹⁸F]FMISO, and other ¹⁸F-labelled compounds wherein production comprises a hydrolytic deprotection step, that improves upon the methods known in the art.

### BRIEF DESCRIPTION OF THE DRAWINGS

FIG. 1 is a schematic diagram of a cassette according to the present invention.

FIG. 2 is a schematic illustration of one way of carrying out the diluting and trapping steps comprised in the method of the present invention, as described in more detail in Example 1.

FIG. 3 is a workflow diagram of showing how the method of the present invention may be carried out and is described in more detail in Example 1.

### SUMMARY OF THE INVENTION

The present invention provides an improved method to prepare an ¹⁸F-labelled compound where the synthesis comprises a hydrolytic deprotection step. Specifically, the method of the invention permits neutralisation of an acidic or basic crude product without using any neutralising chemicals. Instead, the product is trapped on an SPE column and then thoroughly rinsed with water. As a consequence of this process simplification, the method of the invention can more readily be carried out on an automated synthesiser. In addition to the radiofluorination method of the invention, the present invention provides a cassette designed to carry out the method on an automated synthesiser.

### DETAILED DESCRIPTION OF PREFERRED EMBODIMENTS

The present invention therefore provides in one aspect a method comprising:
(i) labelling a protected precursor compound with ¹⁸F;
(ii) deprotecting the ¹⁸F-labelled compound obtained in step (i) by hydrolysis;
(iii) diluting the deprotected ¹⁸F-labelled compound obtained in step (ii) with water;
(iv) trapping the deprotected ¹⁸F-labelled compound on a solid-phase extraction (SPE) column by passing the diluted solution obtained in step (iii) through said column;
(v) eluting the deprotected ¹⁸F-labelled compound obtained in step (iv) from the SPE column;
   with the proviso that no neutralising step is carried out following the deprotection step.

An “¹⁸F-labelled compound” in the context of the present invention is a chemical compound comprising at least one ¹⁸F atom. Preferably, an ¹⁸F-labelled compound of the present invention comprises only one ¹⁸F atom.

The term “labelling” in the context of the present invention refers to the radiochemical steps involved to add ¹⁸F to a compound. The precursor compound is reacted with a suitable source of ¹⁸F to result in the ¹⁸F-labelled compound. A “suitable source of ¹⁸F” is typically either ¹⁸F-fluoride or an ¹⁸F-labelled synthon. ¹⁸F-fluoride is normally obtained as an aqueous solution from the nuclear reaction ¹⁸O(p,n)¹⁸F. In order to increase its reactivity and to avoid hydroxylated by-products resulting from the presence of water, water is typically removed from ¹⁸F-fluoride prior to the reaction, and fluorination reactions are carried out using anhydrous reaction solvents (Aigbirhio et al 1995 J Fluor Chem; 70: 279-87). The removal of water from ¹⁸F-fluoride is referred to as making “naked” ¹⁸F-fluoride.

A further step that is used to improve the reactivity of ¹⁸F-fluoride for radiofluorination reactions is to add a cationic counterion prior to the removal of water. Suitably, the counterion should possess sufficient solubility within the anhydrous reaction solvent to maintain the solubility of the ¹⁸F-fluoride. Therefore, counterions that are typically used include large but soft metal ions such as rubidium or caesium, potassium complexed with a cryptand such as Kryptofix™, or tetraalkylammonium salts, wherein potassium complexed with a cryptand such as Kryptofix™, or tetraalkylammonium salts are preferred.

The term “precursor” refers to a compound that when reacted with a suitable source of ¹⁸F results in the desired ¹⁸F-labelled compound. The term “protected” refers to the presence of one or more protecting groups on the precursor whose presence is required for site-directed incorporation of ¹⁸F. The terms “protecting group” and “deprotecting” are well-known in the art. The use of protecting groups is described in ‘Protective Groups in Organic Synthesis’, by Greene and Wuts (Fourth Edition, John Wiley & Sons, 2007). The step of deprotecting is typically carried out by hydrolysis, either using an acid or a base. The deprotecting step of the present invention is preferably carried out by acid hydrolysis.

The term “diluting” is well-known in the art and refers to the process of reducing the concentration of a solute in solution by mixing with more solvent. In the context of the present invention the solvent used in the diluting step is water. The purpose of the diluting step is to increase the polarity of the reaction mixture in order to permit high and reproducible trapping of the product on an apolar (also commonly termed “reverse-phase”) SPE column.

The term “trapping” in the present invention refers to the retention of the deprotected ¹⁸F-labelled compound on the SPE column by interactions between the deprotected ¹⁸F-labelled compound and the sorbent of the SPE column. These interactions are solvent-dependent.

The term “solid-phase extraction” (SPE) refers to the chemical separation technique that uses the affinity of solutes dissolved or suspended in a liquid (known as the mobile phase) for a solid through which the sample is passed (known as the stationary phase or sorbent) to separate a mixture into desired and undesired components. The result is that either the desired analytes of interest or undesired impurities in the sample are retained on the sorbent, i.e. the trapping step as defined above. The portion that passes through the sorbent is collected or discarded, depending on whether it contains the desired analytes or undesired impurities. If the portion retained on the sorbent includes the desired analytes, they can then be removed from the sorbent for collection in an additional step, in which the sorbent is rinsed with an appropriate eluent. The sorbent is typically packed between two porous media layers within an elongate cartridge body to form the “solid-phase extraction (SPE) column”. High-performance liquid chromatography (HPLC) is specifically excluded from the definition of SPE in the context of the present invention.

The term “neutralising” as used herein refers to the process of adjusting the pH of a solution to bring it back to pH 7, or as close as possible to pH 7. Therefore, an acidic solution can be neutralised by adding a suitable amount of an alkali such as NaOH, and an alkaline solution can be neutralised by adding a suitable amount of an acid such as HCl.

The term “eluting” refers to the process of removing the desired compound from the SPE column by passing a suitable solvent through the column. The suitable solvent for eluting is one in which the interactions between the sorbent of the SPE column and the desired compound are broken thereby allowing the compound to pass through the column and be collected.

In the method of the present invention, a distinct neutralisation step is not carried out. Rather, the step of diluting serves both to bring the pH to neutrality and to prepare the reaction mixture for SPE purification. As compared to the prior art methods, the method of the present invention is therefore simplified by removal of the neutralisation step, which makes the method more straightforward to carry out and to automate.

The method of the invention may be applied to the synthesis of any ¹⁸F-labelled PET tracer that comprises ¹⁸F labelling of a precursor compound that comprises protecting groups and subsequent removal of the protecting groups by acid or alkaline hydrolysis.

Non-limiting examples of such ¹⁸F-labelled PET tracer include ¹⁸F-fluorodeoxyglucose (¹⁸F-FDG), 6-[¹⁸F]-L-fluorodopa (¹⁸F-FDOPA), ¹⁸F-fluorothymidine (¹⁸F-FLT), 1-H-1-(3-[¹⁸F]fluoro-2-hydroxypropyl)-2-nitroimidazole (¹⁸F-FMISO), ¹⁸F-1-(5-fluoro-5-deoxy-α-arabinofuanosyl)-2-mitroimidazole (¹⁸F-FAZA), 16-α-[¹⁸F]-fluoroestradiol (¹⁸F-FES), and 6-[¹⁸F]-fluorometarminol (¹⁸F-FMR). Said ¹⁸F-labelled compound is preferably ¹⁸F-fluorodeoxyglucose (¹⁸F-FDG), 6-[¹⁸F]-L-fluorodopa (¹⁸F-FDOPA), ¹⁸F-fluorothymidine (¹⁸F-FLT), or ¹⁸F-fluoromisonidazole (¹⁸F-FMISO), and most preferably ¹⁸F-fluorothymidine (¹⁸F-FLT) or ¹⁸F-fluoromisonidazole (¹⁸F-FMISO). The known synthesis of each of these PET tracers includes a deprotection step and a neutralisation step (see for example chapters 6 and 9 of “Handbook of Radiopharmaceuticals” 2003; Wiley: by Welch and Redvanly, and chapter 8 of “Basics of PET Imaging, 2^{nd} Edition” 2010; Springer: by Saha). The method of the invention is carried out to obtain any of these PET tracers in purified form in a straightforward manner by omitting the neutralisation step and carrying out the diluting, trapping and eluting steps as defined herein.

Examples of PET tracers which may be synthesised by the method of this aspect of the present invention include [¹⁸F]-fluorodeoxyglucose ([¹⁸F]-FDG), [¹⁸F]-fluorodihydroxyphenylalanine ([¹⁸F]-F-DOPA), [¹⁸F]-fluorouracil, [¹⁸F]-1-amino-3-fluorocyclobutane-1-carboxylic acid ([¹⁸F]-FACBC), [¹⁸F]-altanserine, [¹⁸F]-fluorodopamine, 3′-deoxy-3′-¹⁸F-fluorothymidine [¹⁸F-FLT] and [¹⁸F]-fluorobenzothiazoles.

The structures of various ¹⁸F-labelled protected precursor compounds obtained in step (i) of the method of the present invention are as follows (wherein P¹ to P⁴ are each independently hydrogen or a protecting group):

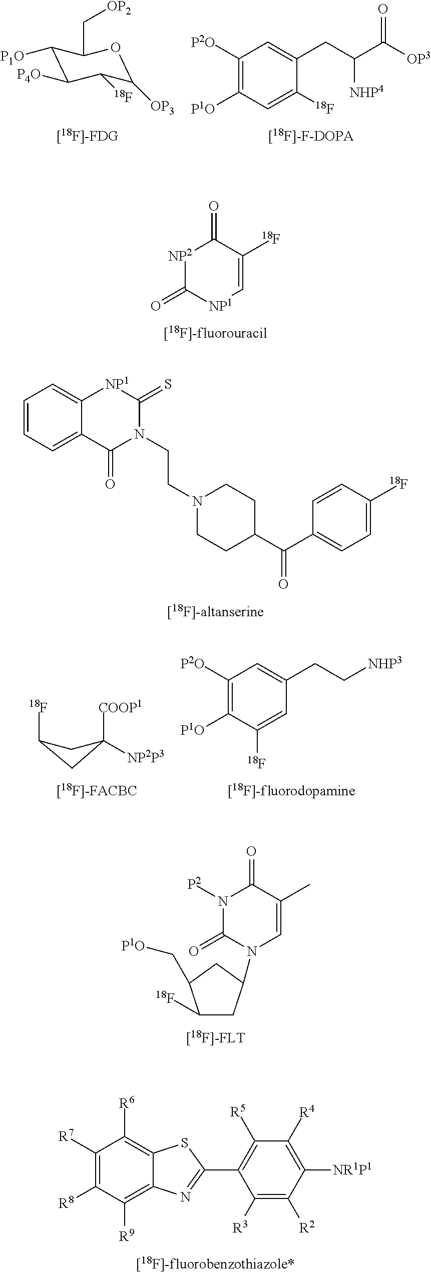

*R¹ is selected from hydrogen, C₁₋₆ alkyl, C₁₋₆ hydroxyalkyl, and C₁₋₆ haloalkyl; R² to R⁹ are independently selected from hydrogen, halo, C₁₋₆ alkyl, C₁₋₆ haloalkyl, C₁₋₆ hydroxyalkyl, C₁₋₆ alkoxy, C₁₋₆ haloalkoxy, hydroxy, cyano, and nitro.

In one embodiment, the method of the invention is used for the synthesis of ¹⁸F-FMISO:

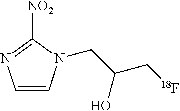 [SMILES: O=[N+]([O-])c1nccn1CC(O)C[18F]]

When ¹⁸F-FMISO is the ¹⁸F-labelled compound obtained by the method of the present invention, a preferred protected precursor compound is a compound of Formula I:

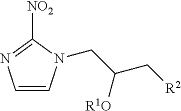

wherein:
R¹ is a protecting group for the hydroxyl function; and,
R² is a leaving group.

R₁ of Formula I is preferably selected from acetyl, benzoyl, dimethoxytrityl (DMT), β-methoxyethoxymethyl ether (MEM), methoxymethyl ether (MOM), and tetrahydropyranyl (THP), and is most preferably THP.

R² of Formula I is a leaving group, wherein the term “leaving group” refers to a moiety suitable for nucleophilic substitution and is a molecular fragment that departs with a pair of electrons in heterolytic bond cleavage. R² is preferably selected from Cl, Br, I, tosylate (OTs), mesylate (OMs) and triflate (OTf), most preferably selected from OTs, OMs and OTf, and is most especially preferably OTs.

A most preferred precursor compound for the synthesis of ¹⁸F-FMISO is 1-(2′-nitro-1′-imidazolyl)-2-O-tetrahydropyranyl-3-O-tosyl-propanediol, i.e. a compound of Formula I wherein R¹ is tetrahydropyranyl and R² is OTs.

In a preferred embodiment of the invention, the diluting step comprises:
(a) adding a first volume of water to said deprotected ¹⁸F-labelled compound to obtain a first diluted solution, and,
(b) adding subsequent volumes of water to aliquots of said first diluted solution to obtain subsequent diluted solutions.

It is intended that the diluting step will result in a reaction mixture having a polarity suitable to permit high and reproducible trapping on an apolar SPE column. Ideally, the diluted reaction mixture should not have more than around 10-15% organic solvent in water in order to achieve this aim. Aliquots of the diluted solution are passed through the SPE column so as to trap the deprotected ¹⁸F-labelled compound onto the column. Optionally, once all the diluted solutions has been passed through the SPE column, an additional step of washing the column with water may be carried out prior to the eluting step.

Preferably, the eluting step is carried out using a solution of aqueous ethanol. In the case of ¹⁸F-FMISO, it is preferred that the eluting step is carried out with an aqueous ethanol solution comprising 2-20% ethanol, most preferably 5-10% ethanol.

The sorbent of the SPE column for the present invention can be any silica- or polymeric-based apolar sorbent. Non-limiting examples of suitable apolar SPE columns include polymer-based Oasis HLB or Strata X SPE columns, or silica-based C2, C4, C8, C18, tC18 or C30 SPE columns. The SPE column of the invention is preferably selected from Oasis HLB, tC18, and Strata X.

¹⁸F-labelled PET tracers are now often conveniently prepared on an automated radiosynthesis apparatus. Therefore, in a preferred embodiment, the method of the present invention is an automated synthesis. The term “automated synthesis” refers to a chemical synthesis that is performed without human intervention. In other words, it refers to a process that is driven and controlled by at least one machine and that is completed without the need of manual interference.

There are several commercially-available examples of such apparatus, including Tracerlab™ and Fastlab™ (GE Healthcare Ltd). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. The automation of synthesis of PET tracers performed on a synthesiser platform is limited by the number of available reagent slots. The method of the present invention permits a reduction in the number of chemicals required by removing the neutralising agent.

In another aspect, the present invention provides a cassette for carrying out the method of the invention, said cassette comprising:
(i) a vessel containing said protected precursor compound as defined herein;
(ii) means for eluting the vessel containing said protected precursor compound with a suitable source of ¹⁸F as defined herein;
(iii) means for deprotecting the ¹⁸F-labelled compound obtained following elution of the vessel containing said protected precursor compound with a suitable source of ¹⁸F; and,
(iv) an SPE column as defined herein suitable for trapping the deprotected ¹⁸F-labelled compound;
   with the proviso that a vessel containing a neutralisation agent suitable for neutralising the pH of said deprotected ¹⁸F-labelled compound is neither comprised in or in fluid connection with said cassette.

In the context of the cassette of the invention, a “neutralising agent” is an acidic or an alkaline solution designed to neutralise the pH of, respectively an alkaline or an acidic solution comprising deprotected labelled ¹⁸F-labelled compound.

All the suitable, preferred, most preferred, especially preferred and most especially preferred embodiments of the precursor compound of Formula Ia, ¹⁸F-fluoride and the SPE cartridges that are presented herein in respect of the method of the invention also apply to the cassette of the invention.

The cassette of the invention may furthermore comprise:
(iv) an ion-exchange cartridge for removal of excess [¹⁸F]-fluoride.

### BRIEF DESCRIPTION OF THE EXAMPLES

Example 1 describes how ¹⁸F-FMISO was obtained according to the method of the invention.

### LIST OF ABBREVIATIONS USED IN THE EXAMPLES

EtOH ethanol
¹⁸F⁻ fluoride
¹⁸F-FMISO 1-H-1-(3-[¹⁸F]fluoro-2-hydroxypropyl)-2-nitroimidazole
ID internal diameter
NITTP 1-(2′-Nitro-1′-imidazolyl)-2-O-tetrahydropyranyl-3-O-toluenesulfonyl-propanediol
MeCN acetonitrile
QMA quaternarymethylammonium
THP tetrahydropyranyl

### EXAMPLES

### Example 1

### Synthesis of ¹⁸F-FMISO

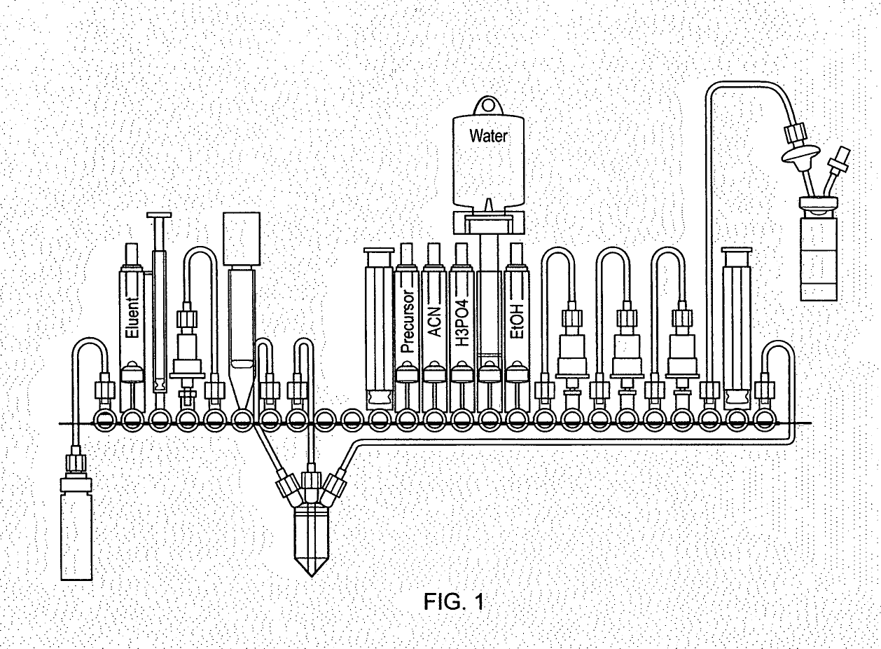

A cassette as illustrated in FIG. 1 was fitted to a FASTlab synthesiser (GE Healthcare).

[¹⁸F]Fluoride was supplied from GE Healthcare on a GE PETrace cylcotron. The initial activity was transferred via the activity inlet of the FASTlab cassette using vacuum. The activity was transferred from the activity inlet to the (pre-treated) QMA cartridge where the [¹⁸F] was trapped and the water passed through to the ¹⁸O water recovery vial, using a combination of N₂ to push and vacuum to pull.

After the transfer of the eluent containing the ¹⁸F− activity into the reaction vessel, the solvents were evaporated until dryness. During the drying process, a small amount of acetonitrile (80 μl) was added to the reaction vessel. The evaporation was carried out with heating under nitrogen flow and under vacuum.

The 1-(2′-Nitro-1′-imidazolyl)-2-O-tetrahydropyranyl-3-O-toluenesulfonyl-propanediol precursor (also called NITTP) was added to the dry residue. Nucleophilic substitution at 110° C. was carried out in the closed reaction vessel, in which the tosylate group of the precursor was replaced by the ¹⁸F− ions. After labelling, the solution is cooled down to 60° C.

The tetrahydropyranylated (THP) compound was converted into ¹⁸F-FMISO by removing the THP protecting group. This deprotection was carried out in the reaction vessel at 90° C. by means of 1 ml of 0.6M H₃PO₄ for about 5 min. This acid concentration was obtained by dilution of 360 μl 2.29M H₃PO₄ with ≈840 μl water. The resulting ¹⁸F-FMISO was obtained in an organic/water mixture. The organic solvent (MeCN) was removed by flushing nitrogen through right hand side connector combined with vacuum (−10 kPa (−100 mBar)) during 8 minutes at 90° C.

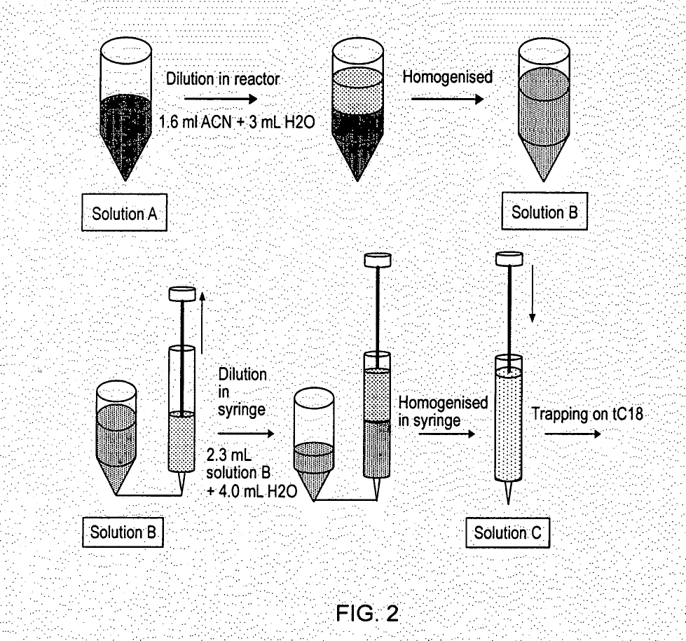

The crude FMISO was mixed in a syringe with 3.5 ml of water, and sent back to the reaction vessel. This solution (B) was then diluted with water in 3 portions. 1.5 ml of this solution (B) was diluted with 5.0 ml of water (solution C) and then passed through the reverse phase cartridge (Oasis® HLB). This operation was done 3 times with the remaining solution in the reaction vessel. The FMISO was trapped onto the cartridge. Solvents, unreacted ¹⁸F⁻ ions and impurities were washed off into the external waste bottle with 7 ml of water. FIG. 2 is a schematic diagram of this dilution and trapping process.

The trapped FMISO was rinsed prior the elution with a full syringe of water (≈7 ml). The elution of the FMISO was performed by dilution of absolute ethanol with water to a ratio of 5 to 6% of EtOH. This dilution was performed in the middle syringe by withdrawing ≈350 μl of EtOH first then about 6.5 ml of water and repeated 3 times. The FMISO was eluted from the Oasis® HLB cartridge trough an acidic alumina light cartridge to the product collection vial.

At the end of the elution, 2 full syringes of nitrogen were flushed trough the transfer tube followed by 30 sec of direct nitrogen flush (HF; 100 kPa (1000 mbar)) in order to allow a transfer trough a 15 m long tubing (min ID: 1 mm).

Non polar by-products were retained on the Oasis® HLB cartridge and the polar, such as last traces of unreacted ¹⁸F⁻, on the Alumina. The solution was finally passed through a vented 0.22 μm filter.

The final volume of ¹⁸F-FMISO was 20 mL±0.5 mL.

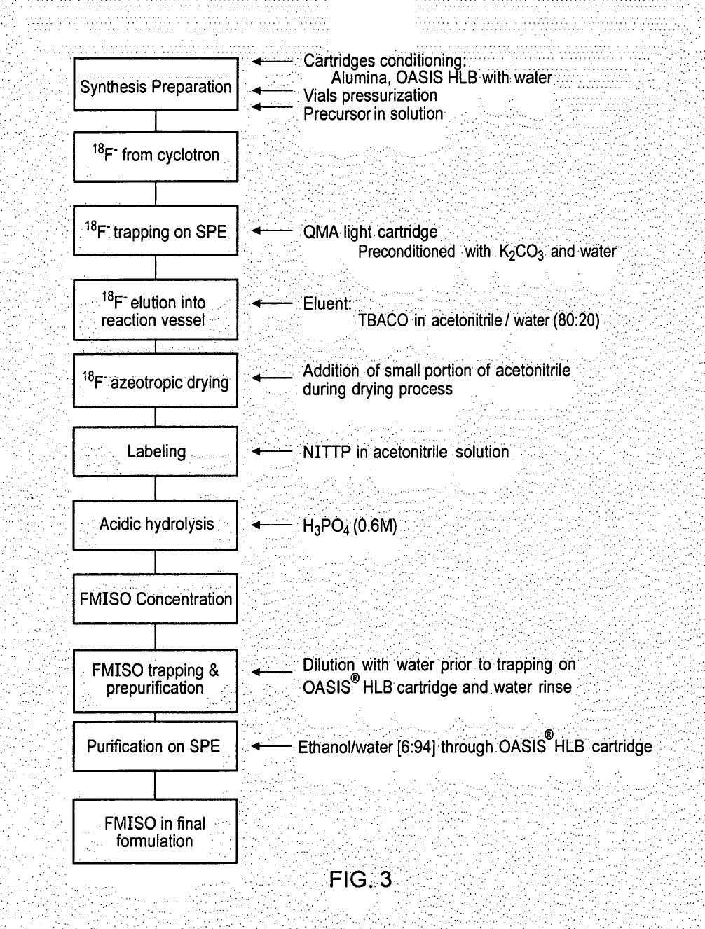

A schematic of the entire process is set out in FIG. 3. The process took less than 57 minutes in total and resulted in uncorrected yields of around 35%.

What is claimed is:

1. A method comprising the steps of:
(i) labelling a protected precursor compound with ¹⁸F;
(ii) deprotecting the ¹⁸F-labelled compound obtained in step (i) by hydrolysis;
(iii) diluting the deprotected ¹⁸F-labelled compound obtained in step (ii) with water;
(iv) trapping the deprotected ¹⁸F-labelled compound on a solid-phase extraction (SPE) column by passing the diluted solution obtained in step (iii) through said column;
(v) eluting the deprotected ¹⁸F-labelled compound from the SPE column;
with the proviso that no neutralising step is carried out following the deprotection step.

2. The method as defined in claim 1 wherein said deprotecting step (ii) is carried out by acid hydrolysis.

3. The method as defined in claim 1 wherein said ¹⁸F-labelled compound is ¹⁸F-fluorodeoxyglucose (¹⁸F-FDG), 6-[¹⁸F]-L-fluorodopa (¹⁸F-FDOPA), ¹⁸F-fluorothymidine (¹⁸F-FLT), ¹⁸F-fluoromisonidazole (¹⁸F-FMISO), ¹⁸F-1-(5-fluoro-5-deoxy-α-arabinofuanosyl)-2-mitroimidazole (¹⁸F-FAZA), 16-α-[¹⁸F]-fluoroestradiol (¹⁸F-FES), or 6-[¹⁸F]-fluorometarminol (¹⁸F-FMR).

4. The method as defined in claim 1 wherein said ¹⁸F-labelled compound is ¹⁸F-fluorodeoxyglucose (¹⁸F-FDG), 6-[¹⁸F]-L-fluorodopa (¹⁸F-FDOPA), ¹⁸F-fluorothymidine (¹⁸F-FLT), or ¹⁸F-fluoromisonidazole (¹⁸F-FMISO).

5. The method as defined in claim 1 wherein said ¹⁸F-labelled compound is ¹⁸F-fluorothymidine (¹⁸F-FLT) or ¹⁸F-fluoromisonidazole (¹⁸F-FMISO).

6. The method as defined in claim 1 wherein said ¹⁸F-labelled compound is 1-H-1-(3-[¹⁸F]fluoro-2-hydroxypropyl)-2-nitroimidazole (¹⁸F-FMISO):

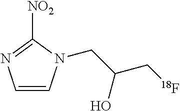 [SMILES: O=[N+]([O-])c1nccn1CC(O)C[18F]]

7. The method as defined in claim 6 wherein said protected precursor compound is a compound of Formula I: 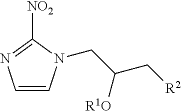
wherein:
R¹ is a protecting group for the hydroxyl function; and,
R² is a leaving group.

8. The method as defined in claim 1 wherein said diluting step further comprises:
(a) adding a first volume of water to said deprotected ¹⁸F-labelled compound to obtain a first diluted solution, and,
(b) adding subsequent volumes of water to aliquots of said first diluted solution to obtain subsequent diluted solutions.

9. The method as defined in claim 1 wherein said SPE cartridge is selected from Oasis HLB, tC18, and Strata X.

10. The method as defined in claim 1 wherein each step is automated.

11. A cassette for carrying out the method as defined in claim 1, said cassette comprising:
(i) a vessel containing said protected precursor compound as defined in claim 7;
(ii) means for eluting the vessel containing said protected precursor compound with a suitable source of ¹⁸F;
(iii) means for deprotecting the ¹⁸F-labelled compound obtained following elution of the vessel containing said protected precursor compound with a suitable source of ¹⁸F; and,
(iv) an SPE column as defined in claim 9 suitable for trapping the deprotected ¹⁸F-labelled compound;
with the proviso that a vessel containing a neutralisation agent suitable for neutralising the pH of said deprotected ¹⁸F-labelled compound is neither comprised in or in fluid connection with said cassette.
